(19) United States
(12) Plant Patent Application Publication
(10) Pub. No.: US 2018/0255674 P1
(43) Pub. Date: Sep. 6, 2018

(54) STRAWBERRY PLANT CALLED 'SHARKTOOTH'

(50) ;

(71) Applicant: Olivia Louise Brough, Powell, OH (US)

(72) Inventor: Olivia Louise Brough, Powell, OH (US)

(21) Appl. No.: 15/530,837

(22) Filed: Mar. 6, 2017

(51) Int. Cl. A01H 6/74

(52) U.S. Cl. PLT/209

(57) ABSTRACT

A strawberry plant named ‘Sharktooth’ having unique flower and berry colors, among other unique characteristics. The flowers of ‘Sharktooth’ are a diluted pink and the fruit is white with pink undertones and red achenes, a mix of characteristics from its parents ‘White D’ and ‘Tarpan’. The fruit flavor is pleasant and unique with flavor similar to ‘White D’ but with less acidity and added sweetness similar from its ‘Tarpan’ parent.

### CROSS-REFERENCE TO RELATED APPLICATION

This application makes no priority claim.

Genus and species: This strawberry cultivar of this invention is botanically identified as fragaria x ananassa ‘Sharktooth’.

### BACKGROUND AND SUMMARY OF THE INVENTION

‘Sharktooth’ originated from a cross performed in 2015 between the cultivar ‘Tarpan’ (fragaria x ananassa) and ‘White D’ (fragaria x ananassa). ‘Sharktooth’ was first crossed in 2015 in Powell, Ohio where it was propagated asexually by runners. The plant has been named ‘Sharktooth’ for introduction. Asexual propagules from this original source have been tested in personal growing fields starting in 2016. The properties of this variety were found to be transmissible by such asexual reproduction.

This invention relates to a new and distinctive cultivar designated ‘Sharktooth’. ‘Sharktooth’ Is day-neutral (ever-bearing) cultivar with a diluted light pink color flower and white colored fruit with pink undertones and red achenes. ‘Sharktooth’ flowers get their coloring from a combination of ‘Tarpan's’ deep pink/magenta color and ‘White D's’ white color. ‘Sharktooth’ fruit likewise gets is coloring from a combination of ‘Tarpan's’ bright red fruit and ‘White D's’ white color. ‘Sharktooth’ fruit retains the red achenes from ‘White D’. ‘Sharktooth’ fruit has an elongated pyramidal shape, which is a mix of its parents' characteristics. ‘Sharktooth’ berries have a pleasant and unique taste compared to its parents.

### BRIEF DESCRIPTION OF THE DRAWINGS

The following figures depict various characteristics of the ‘Sharktooth’ cultivar:

FIG. 1 shows the general flowering characteristics of the plant;

FIG. 2A shows the general flowering characteristics of the plant;

FIG. 2B shows the general flowering characteristics of the plant;

FIG. 2C shows the general flowering characteristics of the plant;

FIG. 2D shows the general flowering characteristics of the plant;

FIG. 3 shows the general fruiting characteristics of the plant;

FIG. 4 shows the general fruiting characteristics of the plant;

FIG. 5A shows the general fruiting characteristics of the plant;

FIG. 5B shows the general fruiting characteristics of the plant;

FIG. 5C shows the general fruiting characteristics of the plant;

FIG. 6 shows several typical plants;

FIG. 7 shows several typical plants;

FIG. 8 shows the general fruiting characteristics of the plant; and

FIG. 9 shows several typical plants.

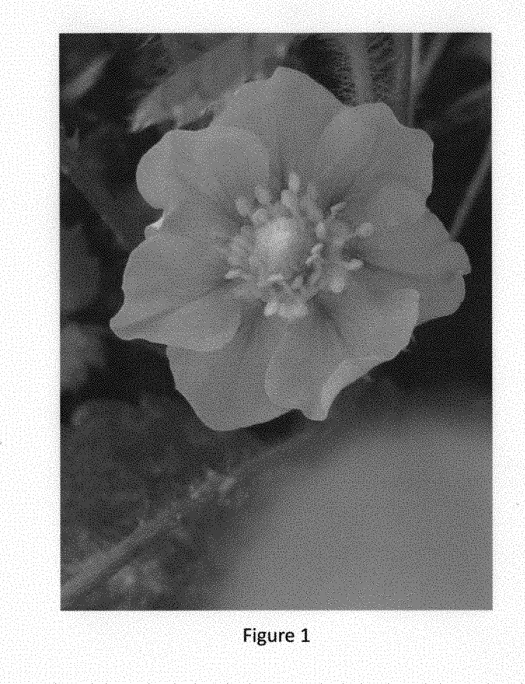

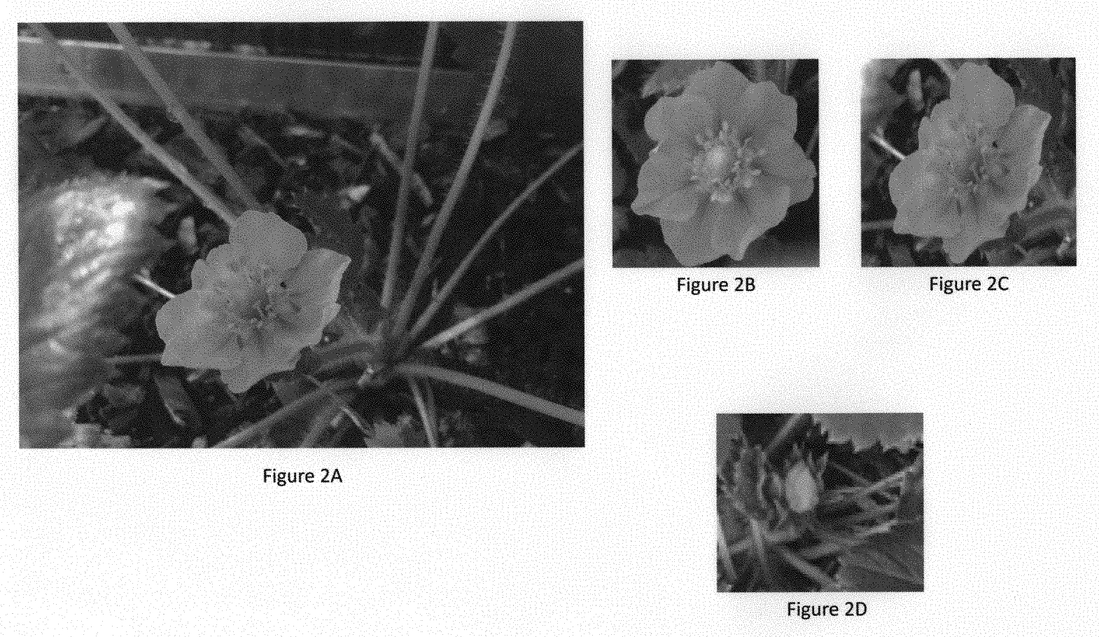

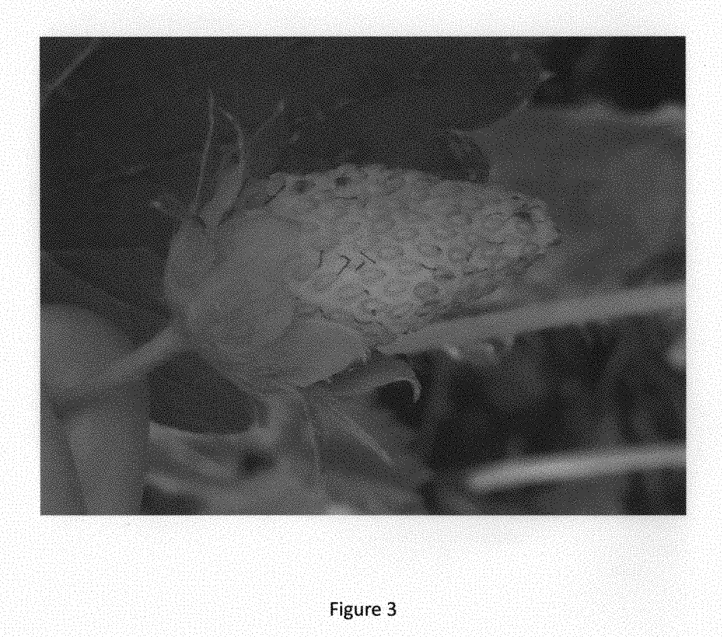

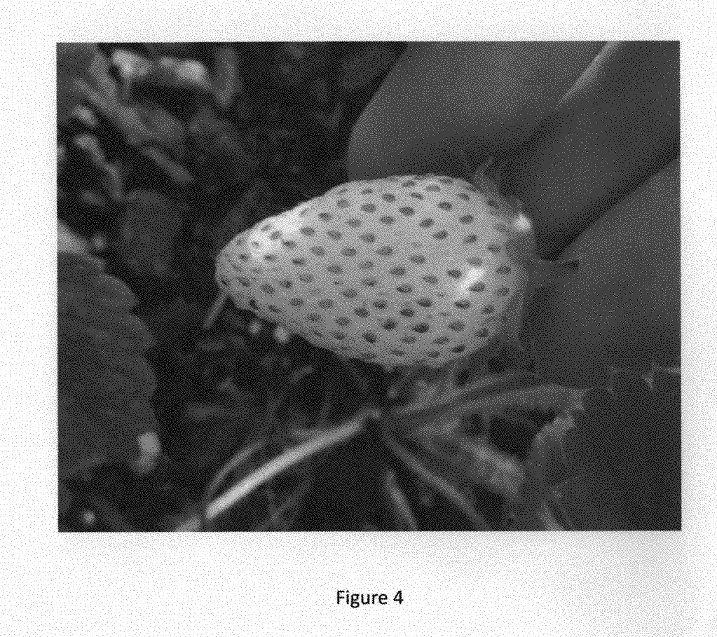

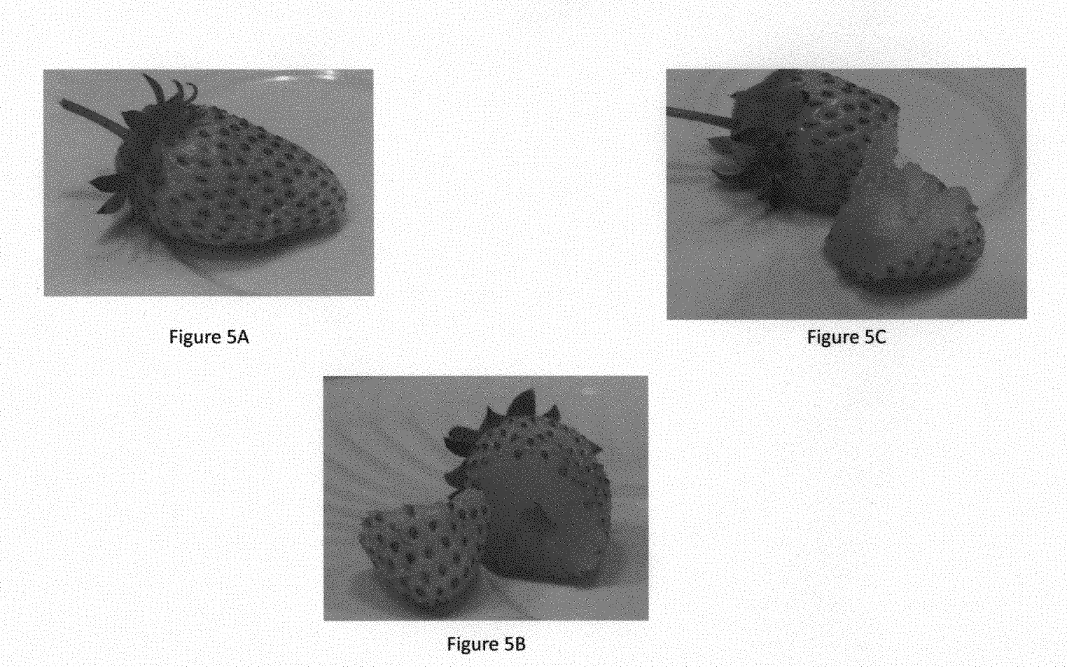

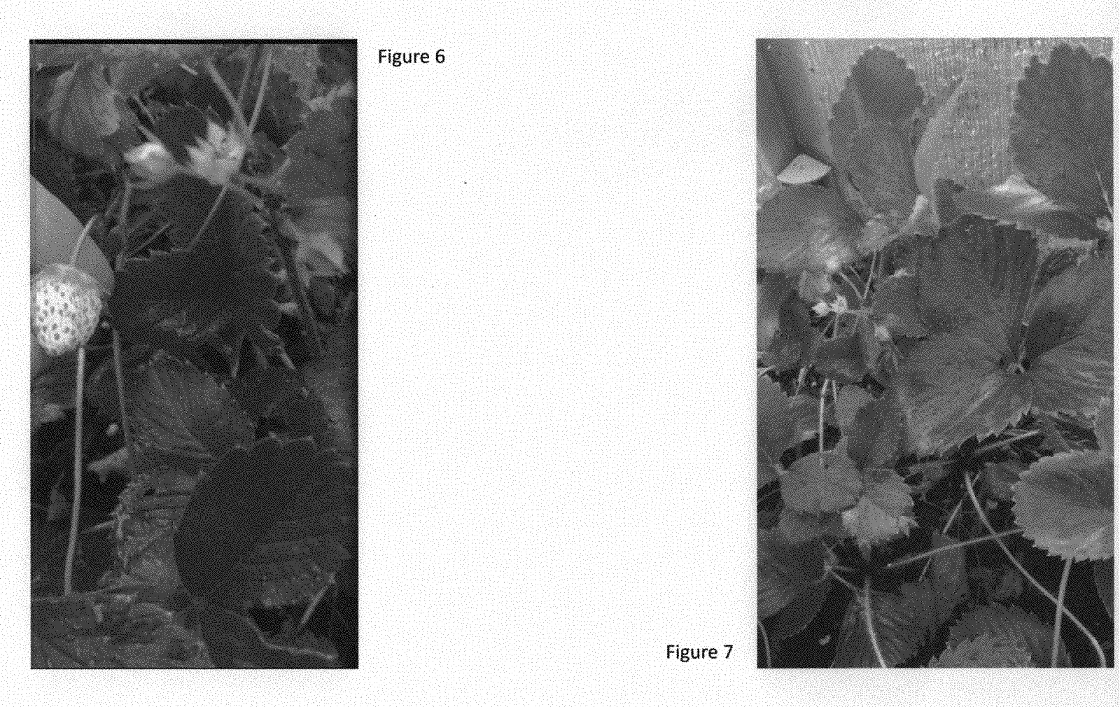

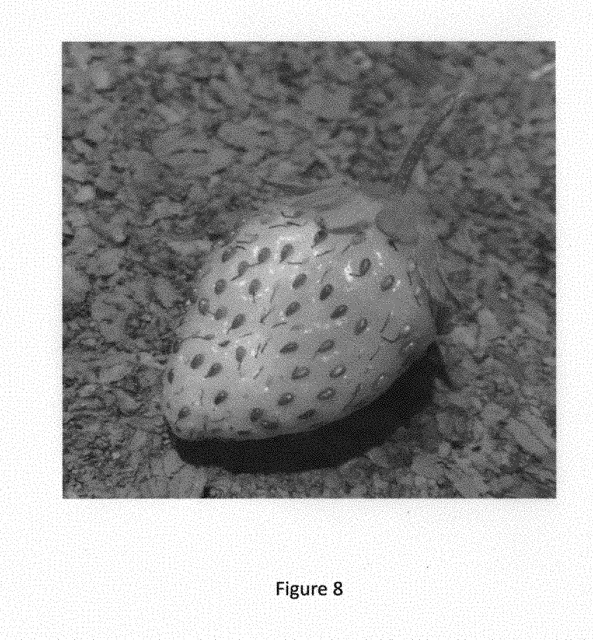

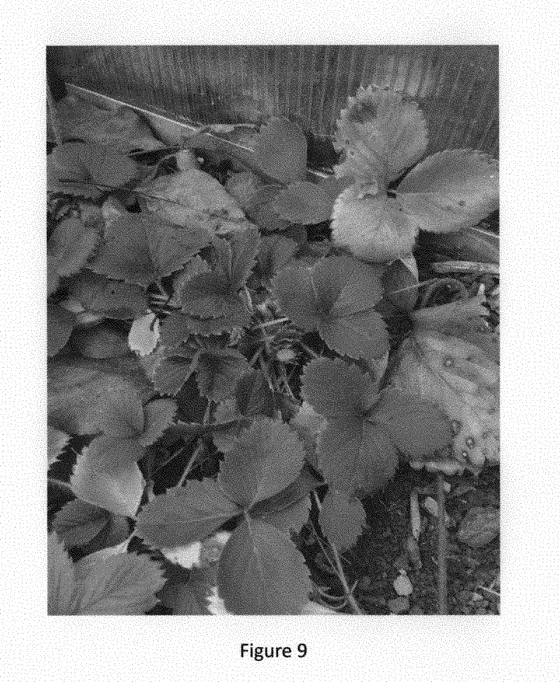

### DETAILED DESCRIPTION OF EXEMPLARY EMBODIMENT(S)

The invention relates to a new and distinctive day-neutral type cultivar designated as ‘Sharktooth’. ‘Sharktooth’ is typical of day-neutral strawberry plants and produces fruit regardless of day length when treated appropriately in humid or arid, subtropical climates. ‘Sharktooth’ has the following characteristics relative to its ‘Tarpan’ parent, its flowers retain pink-ish hues (though diluted by its cross with the ‘White D’ parent), elongated narrow berry shape (though more pyramidal because of its cross with the conic ‘White D’ parent), and pink undertones in its fruit (though dominantly white from its cross with the ‘White D’ parent). ‘Sharktooth’ has the following characteristics relative to its ‘White D’ parent, its flowers have a diluted light pink hue (the pink tones retained by its cross with the pink/magenta ‘Tarpan’ parent) and its fruit are white (though diluted with pink undertones from its red ‘Tarpan’ parent) and contain red achenes. ‘Sharktooth’ berries have a pleasant and unique taste, combining the most appealing characteristics of both of its parents.

Plants and foliage: Fruiting plants of ‘Sharktooth’ are similar in morphology to ‘Tarpan’ and ‘White D’ parents. The habit is semi-erect to prostrate, usually semi-erect. ‘Sharktooth's’ habit of growth is similar to its parents and generally has a maximum height of six inches (6″), with small leaves and inflorescence even with or below the leaf canopy. This is similar to both of its parents, ‘Tarpan’ and ‘White D’.

Flowering, fruiting, fruit, and production characteristics: ‘Sharktooth’ is similar to other day-neutral plants (e.g., ‘Tarpan’) in that it will flower and fruit independently of day length, given appropriate temperature and horticultural conditions. This is unlike its parent ‘White D’, which is a June-bearing plant that tends to produce flowers on new runners in later summer.

The flowering color for ‘Sharktooth’ can vary but is typically a light pink, which is easily distinguished from the ‘White D’ (white flower) and the ‘Tarpan’ (deep pink/magenta). ‘Sharktooth’ flower colors can vary, but when viewed in natural daylight typically range between Pantone Colors Magenta0521C and 183C at the lightest to 7433C and 198C at its darkest, with Magenta0521C being the dominant color. For contrast, ‘Tarpan’ flower colors can vary but typically range between Pantone Colors 236C at the lightest to 213C at the darkest, and 212C as the dominant color. The closeness of the ‘Tarpan’ flower's darkest color to its dominant color demonstrates that the magenta tones dominate the ‘Tarpan’ flower, making it clearly more magenta in color than ‘Sharktooth’ which is a pale pink. Again, for contrast, ‘White D’ flowers are white, like many other strawberry varieties. Thus, ‘Sharktooth’ flowers demonstrate incomplete dominance of the flower color genes of its parents. Instead of being white or magenta, ‘Sharktooth’ flowers are a blend of each and result in a pale pink hue.

‘Sharktooth’ flowers typically have seven (7) petals and are approximately one inch (1″) in diameter with semi-double petals that slightly overlap each other. ‘Tarpan’, for contrast, has flowers that are approximately one to one-and-a-half inches (1″ to 1.5″) in diameter that are also semi-double. ‘White D’ flowers are approximately three quarters of an inch (¾″) in diameter.

‘Sharktooth’ stalks typically have three to four (3-4) flowers, while ‘Tarpan’ typically have five (5) flowers and ‘White D’ typically have three to five (3-5) flowers. The calyx/sepals for ‘Sharktooth’ are visible where the corolla/petals meet with the receptacle in ‘Sharktooth’. The petals are darkly marked at this spot, similar to ‘Tarpan’. However, the ‘Sharktooth’ receptacle is larger than ‘Tarpan’.

‘Sharktooth’ fruit color is similar to ‘White D’, but is slightly whiter and peachier due to pink undertones retained from its ‘Tarpan’ parent. ‘White D’ has green undertones to its white color. Both are in stark contrast to Tarpan' bright red color fruit.

There is little difference in ‘White D's’ color progression from forming to when it is ripe, which remains white with green undertones. However, ‘Sharktooth’ is very white when forming before changing to white with a pinkish blush, resulting in a peachier color when ripe. More specifically, ‘Sharktooth’ fruit, when ripe and viewed in natural light, ranges from Pantone Color 713C to 7506C. In darker, indoor lighting, ‘Sharktooth’ fruit takes on a warm and caramel color ranging from Pantone 1605C to 7511C. This is different from ‘White D’, which is green and cool colored in dark indoor lighting, ranging from Pantone 5845C to 7760C. For contrast, ‘Tarpan’ berries are bright red, having Pantone colors including 7621C, 1785C, 198C, and 7622C, with 7621C being the dominant color.

As another point of contrast, ‘Tarpan’ has gold/brown achenes in contrast to ‘Sharktooth’ and ‘White D, which both have red achenes.

The internal fruit color for ‘Sharktooth’ is lighter than ‘Tarpan’ (mostly bright red) and is substantially the same as ‘White D’ (white).

The fruit shape for ‘Sharktooth’ can vary but is typically an elongated, pyramidal shape approximately one inch (1″) in length. While the length of ‘Sharktooth’ fruit is similar to ‘White D’ and ‘Tarpan’, ‘Sharktooth’ fruit is easily distinguished in shape from ‘White D’ (shortened and globose to globose conical) and ‘Tarpan’ (elongated and cylindrical).

‘Sharktooth’ fruit has a preferable taste compared to its parents. ‘White D’ fruit is generally acidic with no typical strawberry flavor and is often referred to as “pineberry” because of its similarity in taste to pineapple. ‘Tarpan’ is a balance of sweet and tart, with an acceptable, typical strawberry flavor. ‘Sharktooth’ on the other hand, has a pleasant and unique taste that is similar to ‘White D’ but without the acidity and also having the added sweetness from its ‘Tarpan’ parent.

The first flower from seed bloomed July 19^{th}. The first berry was ripe 23 days later (August 10th harvest).

‘Sharktooth’ is self-fertile and it can receive pollen from other strawberries of fragaria x ananassa. ‘Sharktooth’ is cold hardy to Zone 5, like its parents.

It is possible that the phenotype may vary somewhat with variations in the environment. Phenotypic features may also vary depending on culture conditions.

What is claimed is:

1. A new and district variety of strawberry plant named ‘Sharktooth’ substantially as herein shown and described.
